(19) United States
(12) Patent Application Publication
(10) Pub. No.: US 2025/0387529 A1
(43) Pub. Date: Dec. 25, 2025

(54) AN UPPER AIR DISINFECTION SYSTEM

(71) Applicant: SIGNIFY HOLDING B.V., EINDHOVEN (NL)

(72) Inventors: ERIK PAUL BOONEKAMP, BUNNIK (NL); PETER JOHANNES MARTINUS BUKKEMS, DEURNE (NL)

(21) Appl. No.: 18/878,678

(22) PCT Filed: Jun. 29, 2023
(86) PCT No.: PCT/EP2023/067769

§ 371 Date: Dec. 24, 2024

(30) Foreign Application Priority Data
Jul. 1, 2022 (EP) 22182545.8

(51) Int. Cl. A61L 9/20

(52) U.S. Cl. CPC A61L 9/20; A61L 2209/12

(57) ABSTRACT

A lighting system (1) configured to disinfect air in an upper part of a space or room, such as a part of a space or room near a ceiling (17) of the space or room, the lighting system (1) comprising a housing (2) comprising a back wall (21) configured for abutment with a mounting surface and a circumferential wall (22) extending from the back wall (21), at least one LED light source (3) configured to, in operation, emit UV light, a reflector (5) configured to be arranged between the back wall (21) and the at least one LED light source (3) such as to reflect the light source light as collimated light in a main issue direction (ID) generally away from the back wall (21), the reflector (5) being an elastically deformable reflective sheet, and the lighting system further comprising a bridge component (6) being elongated in an elongation direction (ED) perpendicular to the main issue direction (ID) and being arranged between the reflector (5) and the at least one LED light source (3) in such a way that the reflector (5) is forced to assume a curved shape around the bridge component (6), said curved shape comprising a parabolic cross section in a plane perpendicular to the elongation direction (ED), wherein a maximum level of stress imposed on the elastically deformable reflective sheet is falling within the elastic regime of the material of the elastically deformable reflective sheet.

### FIELD OF THE INVENTION

The present invention relates to a lighting system, and in particular to a lighting system configured to disinfect air in an upper part of a space or room, such as a part of a space or room near a ceiling of the space or room, by making use of UV light, and in particular UVC light.

As used herein, such a system is also referred to as an “UVC upper air disinfection system”.

The term “elastic regime” as used herein is a well known term within material physics, and refers to the regime in which, when a solid body of a given material is subjected to stress, the material undergoes elastic deformation only. Elastic deformation is a deformation in which the inflicted change in relative positions of points in a solid body disappears when the stress is removed, and thus a deformation from which the solid body is able to return to its original state once the stress is removed.

The term “optical area” as used herein is intended to refer to the area within the lighting system in which interaction between UV light and components of the lighting system occurs. Thus, outside of the optical area, no interaction between UV light and components of the lighting system occurs.

The term “collimated light” as used herein is intended to mean light having a relatively narrow beam angle, for example a FWHM of less than or equal to 15 degrees, such as 10 degrees, or 5 degrees or less, in the direction of the parabolic cross section of the reflector.

### BACKGROUND OF THE INVENTION

The ultraviolet wavelength range is defined as light in a wavelength range from 100 to 380 nm. UV light suitable for disinfection purposes may in general terms be divided into three main types, namely UVA light with a wavelength in the range of 315 to 400 nm, UVB light with a wavelength in the range of 280 to 315 nm and UVC light with a wavelength in the range of 100 to 280 nm. UVC light inactivates both bacteria and viruses but may also be harmful to human beings and other living creatures. UVA light can only be used for killing viruses. Also, the germicidal effect of UV light varies within the spectrum of UV light. Furthermore, different bacteria and viruses may be vulnerable to different wavelengths of UV light.

The ultraviolet wavelength range can in more details be divided into different types of UV light/UV wavelength ranges (Table 1). Different UV wavelengths of radiation may have different properties and thus may have different compatibility with human presence and may have different effects when used for disinfection (Table 1).

TABLE 1
Properties of different types of UV, violet, and NIR wavelength light

| | | | (Relative) | | | | |
|---|---|---|---|---|---|---|---|
| | | | sterilization | | | | |
| | Short | Wavelength | effectiveness | | Safe | Vitamin D | Ozone |
| Name | name | (nm) | Bacteria | Viruses | Radiation | generation | generation |
| Near | NIR | 750-950 | + | +/− | +++ | | |
| Infrared | | | | | | | |
| (deep red) | | | | | | | |
| Violet | V | 380-420 | +/− | − | + | | |
| Ultra- | UV-A | 315-380 | + | − | + | | |
| violet A | | | | | | | |
| Ultra- | UV-B | 280-315 | + | +/− | +/− | + | |
| violet B | | | | | | | |
| Near | Near | 230-280 | ++ | ++ | − | | |
| ultraviolet | UV-C | | | | | | |
| C | | | | | | | |
| Far ultra- | Far | 190-230 | +++ | +++ | + | | +/− |
| violet C | UV-C | | | | | | |
| Extreme | Extreme | 100-190 | +++ | +++ | − | | + |
| ultraviolet | UV-C | | | | | | |
| C | | | | | | | |

Each UV type/wavelength range may have different benefits and/or drawbacks. Relevant aspects may be (relative) sterilization effectiveness, safety (regarding radiation), and ozone production (as result of its radiation). Depending on an application a specific type of UV light or a specific combination of UV light types may be selected and provides superior performance over other types of UV light. UV-A may be (relatively) safe and may inactivate (kill) bacteria but may be less effective in inactivating (killing) viruses. UV-B may be (relatively) safe when a low dose (i.e., low exposure time and/or low intensity) is used, may inactivate (kill) bacteria, and may be moderately effective in inactivating (killing) viruses. UV-B may also have the additional benefit that it can be used effectively in the production of vitamin D in a skin of a person or animal. Near UV-C may be relatively unsafe, but may effectively inactivating, especially kill bacteria and viruses. Far UV may also be effective in inactivating (killing) bacteria and viruses but may be (relatively to other UV-C wavelength ranges) (rather) safe. Far-UV light may generate some ozone which may be harmful for human beings and animals. Extreme UV-C may also be effective in inactivating (killing) bacteria and viruses but may be relatively unsafe. Extreme UV-C may generate ozone which may be undesired when exposed to human beings or animals. In some application ozone may be desired and may contribute to disinfection, but then its shielding from humans and animals may be desired. Hence, in the table “+” for ozone production especially implies that ozone is produced which may be useful for disinfection applications, but may be harmful for humans/animals when they are exposed to it. Hence, in many applications this “+” may actually be undesired while in others, it may be desired. The types of light indicated in above table may in embodiments be used to sanitize air and/or surfaces.

The terms “inactivating” and “killing” with respect to a virus may herein especially refer to damaging the virus in such a way that the virus can no longer infect and/or reproduce in a host cell, i.e., the virus may be (essentially) harmless after inactivation or killing.

Upper room UV disinfection is a relatively simple and effective means of controlling airborne infection and can be cost-effective for many types of facilities (hospitals, offices etc.). The general concept of upper room UV disinfection is well known in the art. The most common approach is to irradiate the upper part of the room with a UVC light source having a strongly asymmetric beam shape. The UVC source, which for instance may be a gas discharge tube or a LED, is located close to the ceiling of a room.

JP 2004 319323 A discloses a bactericidal lamp fitting comprising a lamp and a nearly box-shaped housing that encases the lamp and is provided with an opening on one side thereof for directing light from the lamp obliquely upward. A reflecting plate in a nearly paraboloidal external shape is installed in the housing and extends from the upper end of the opening to near the rear of the lamp to reflect light radiated from the lamp at an upward direction angled at least 5°. A light shielding plate is installed which rises to a height at or above the highest point of the lamp and forms the lower end of the opening such as to reflect light propagating in a generally downward direction upwards.

Generally, lighting systems configured for disinfecting air by use of UVC light are imposed with strict safety limits due to the potential damaging effect of UVC light on humans and other living creatures.

With the current UVC tube systems lamellae are needed to create a narrow beam for upper air such as to meet the safety limits and restrictions. With such lamellae a lot of UVC light is removed. This results in a low optical efficiency of the system. LEDs have the advantage of a small optical area and is easier controlled by a reflector system. This LED system with relatively low UVC power compared to a tube could be much more efficient but still having the same light output on system level. This means that in principle a UVC LED system could be much more efficient compared to a conventional system. To obtain this, however, a more efficient optical system and limits on the use of lamella must be achieved. If using more reflective surfaces there is also a risk that light will scatter or redirect in the wrong direction and could become a safety concern.

Furthermore, an accurate bent sheet metal reflector or effective low cost UVC reflector is needed to enable an efficient optical design. However, with currently existing prototypes there is difficulties in obtaining an accurate bent sheet metal reflector or effective low cost UVC reflector. Model design and deformation tolerance of the reflector prevents obtaining a sufficiently good and safe UVC upper air disinfection system.

Sheet reflectors do have a thickness tolerance and an elastic and plastic deformation limits. The plastic deformation limit in combination with the thickness tolerance causes challenges in obtaining the tolerance needed for the UVC upper air disinfection system to fulfill the safety requirements.

In prior art sheet forming and installation processes the sheet reflector is clamped between two metal parts and then bent in shape. Because the sheet thickness tolerance is in this clamping direction, one gets extra tolerance in the bent angle and defined shape. Trials have shown that this is not sufficient for ensuring that a resulting UVC upper air disinfection system to operate within the safety requirements. Therefore, with such a UVC upper air disinfection system lamellae are still needed.

Currently known stable UVC reflectors are made from sheet aluminum and polished till a very high grade needed for short UVC wavelength is obtained. After polishing a special coating is applied to protect and enhance the surface reflection. This high-grade polishing is very difficult to obtain in different known cost-effective techniques like extrusion, milling, molding etc. Other methods like diamond milling, diamond turning, slumping etc. are expensive for mass manufacturing and with that not sufficiently cost effective in comparison with the current UVC upper air disinfection systems with lamellae.

There is thus a desire for providing a more accurate and safety compliant UVC upper air disinfection system without the need of expensive manufacturing technologies.

Particularly, there is a desire to improve the reflector of such a UVC upper air disinfection system without increasing costs or even with lowering the costs as compared to current UVC upper air disinfection systems with lamellae.

More generally, there is a desire to improve the reflector of a lighting device according to the invention, whether used as a UVC upper air disinfection system or for another use employing infrared or visible light, without increasing costs or even with lowering the costs as compared to current systems, especially such as to obtain narrow beam light and low glare.

EP2959980A1 discloses a modular UV-LED lamp reflector assembly.

### SUMMARY OF THE INVENTION

It is an object of the present invention to overcome this problem, and to provide a UVC upper air disinfection system being more accurate and safety compliant, and which may be manufactured without the need of expensive manufacturing technologies.

It is a further object of the present invention to reduce the size of the optics of the UVC upper air disinfection system.

It is a further object of the present invention to improve the reflector of such a UVC upper air disinfection system without increasing costs or even with lowering the costs as compared to current UVC upper air disinfection systems with lamellae.

According to a first aspect of the invention, this and other objects are achieved by means of a lighting system as set out in the appended set of claims. The lighting system comprising a housing comprising a back wall and a circumferential wall extending from the back wall, at least one LED light source configured to, in operation, emit light source light, a reflector having a reflector width in an elongation direction, the reflector being configured to be arranged between the back wall and the at least one LED light source such as to reflect the light source light as collimated light in a main issue direction generally away from the back wall, where the reflector is an elastically deformable reflective sheet, and where the lighting system further comprises a bridge component having a bridge width in the elongation direction perpendicular to the main issue direction, wherein the light source, the reflector, and the bridge component are arranged in the housing, wherein the bridge component is arranged between the reflector and the at least one LED light source in such a way that the reflector is forced to assume a curved shape around the bridge component, said curved shape comprising a parabolic cross section in a plane perpendicular to the elongation direction, wherein a maximum level of stress imposed on the elastically deformable reflective sheet is falling within the elastic regime of the material of the elastically deformable reflective sheet.

The way the reflector is forced to assume the curved shape can, for example, be explained as follows:
The back wall and the circumferential wall form the housing with a cavity with an open side opposite to the back wall. The bridge component is to be inserted into the cavity. The bridge component has a length somewhat shorter than a first distance between a first pair of opposite portions of the circumferential wall, i.e. side walls;
The reflector initially is arranged at the open side on the circumferential wall. The reflector has a width of about the first distance and the reflector has a length which is larger than a second distance between a second pair of opposite portions of the circumferential wall, i.e. top wall and bottom wall (which are oriented perpendicular to the side walls);
With the reflector being initially arranged on the open side, the bridge portion is inserted into the cavity and thereby pushing the reflector into the cavity. The reflector thus is forced to assume a curved shape around the bridge component by the combined working of the top wall and bottom wall of the circumferential wall, the bridge component and the specified length of the reflector being larger than the second distance between the top wall and bottom wall. Tolerance analyses have shown that there are a number of possible defects associated with bending a sheet material due to the stresses inflicted on the sheet material during bending. These defects include tolerances introduced on the transition between elastic and plastic deformation, sheet thickness tolerance with respect to bending angle, maximum strain value/heat treatment variations of the material, and tool tolerances needed for plastic deformation. These defects combined result in too much light being emitted in undesired directions. In normal applications this is usually not a problem and is optimized sufficiently, especially by using a suitable scattering material or scattering surface, to limit or smoothen light in that direction. With UV applications, however, this becomes a problem due to the safety limits it is mandatory to apply and is the reason to desire a very accurate bent sheet reflector.

The inventors have shown that by providing that the reflector is an elastically deformable reflective sheet, and that the lighting system further comprises a bridge component shaped and configured to be arranged between the reflector and the at least one LED light source in such a way that during assembly of the lighting system the bridge component shapes the reflector by bending the elastically deformable reflective sheet into a parabolic shape while subjecting the elastically deformable reflective sheet to a level of stress falling within the elastic regime of the material of the elastically deformable reflective sheet, such a very accurate bended sheet reflector may be obtained. Thereby, it is ensured that only the elastic principle of deformation, being the most accurate principle of deformation, and setup tolerances is employed. This in turn minimizes the total tolerance chain from optical center to reflective surface. Using only elastic deformation to build up the reflector further ensures that there is no elongation of the material at all, which maintains the highest reflective performance of the material.

Thereby, an improved reflector of a lighting device according to the invention is provided without increasing costs or even with lowering the costs as compared to current systems, especially such as to obtain narrow beam light and low glare. The lighting system could have the feature that the reflector has a focal line in the elongation direction and wherein the light source is extending along the elongation direction on the focal line of the reflector. Thus a further improved reflector of a lighting device according to the invention is provided, especially in relation to control of beam direction and/or to obtain a narrow beam light, such as a beam having light rays that lie mutually parallel in a direction perpendicular to the elongation direction and the main issue direction. This is of particular relevance for, for example, applications where overhead, grazing UV light along a ceiling in crowded rooms is desired or needed.

The lighting system could have the feature that the reflector is bend over at substantially its full reflector width WR around the bridge component over the full bridge width WB of the bridge component, wherein 0.9*WR<=WB<=WR. Thereby a much accurate bending and curved shape of the reflector and hence of an accurate collimated beam is obtained than the curved shape and collimated beam of a reflector obtained by a forced curvature around, for example, a point contact between reflector and bridge component.

In an embodiment, the lighting system is configured to disinfect air in an upper part of a space or room, such as a part of a space or room near a ceiling of the space or room, and wherein the at least one LED light source is configured to, in operation, emit UV and/or violet light source light.

Thereby, an improved reflector of such a UVC upper air disinfection system is provided without increasing costs or even with lowering the costs as compared to current UVC upper air disinfection systems with lamellae, especially because no special tools are needed. For instance, laser cutting or cutting would be sufficient. This tool-less design also enables more flexibility in narrow beam lighting design, and in applications or designs where low or no glare is needed. Further, this enables providing a lighting system with a smaller, more compact, design and a higher optical efficiency.

A further advantage of such a bridge component is that an accurate distance between the reflector and the UV LED light sources is obtained, which adds to the optical performance and efficiency of the lighting system, especially in virtue of using one single length component between the front side of the reflector and the front side of the substrate. In this connection, the front side of the substrate on which the light emitting devices are arranged is to be understood as the side where the at least one LED light source is arranged.

Still further, such a system opens for the possibility of using different more elastic materials next to aluminum for the reflector. For instance, the use of spring steel or a thin sheet of titanium becomes possible. With such materials the size of the optics of the system may be reduced, since the size is limited by the elastic properties of the material of the reflector.

In an embodiment, the circumferential wall comprises an upper wall, a lower wall extending in parallel with the upper wall and two mutually parallel side walls extending between the upper wall and the lower wall, and the curved shape or the parabolic shape into which the elastically deformable reflective sheet is bent follows a curve extending between the upper wall and the lower wall of the housing.

Thereby a properly oriented parabolic reflector is provided for, which in turn further improves the optical performance and efficiency of the lighting system.

In an embodiment, the bridge component comprises two side plates and a holding component, where the two side plates each comprise an upper edge, a lower edge and a front edge extending between the upper edge and the lower edge, the front edge being configured for abutment with the reflector in the assembled condition of the lighting system, the front edge comprising a parabolic curvature such that when the front edge and the reflector are brought into abutment during assembly of the lighting system, the reflector is provided with a parabolic curvature corresponding to that of the front edge, and where the holding component extends between and perpendicular to the side plates midways between the upper edge and the lower edge, and the holding component is configured for abutment with the reflector in the assembled condition of the lighting system.

To keep the elastic reflector in place a construction that keeps a constant tension on the reflector is needed. This is obtained by providing a bridge component as described above in combination with the housing. With such a bridge component it becomes possible to bend the reflector in a perfect parabola during assembly of the lighting system. Further, providing such a bridge component, and in particular such a holding component, ensures that the parabolic reflector is fixed in the correct shape after bending.

Furthermore, such a bridge construction enables obtaining a low glare/low stray light by preventing scattering on edges inside the optical beam. Especially, such a bridge construction ensures that all reflector connections are arranged outside the optical area as defined further below. This in particular prevents UVC light scattering through the safety plane when used in upper air cleaning systems.

Compared to the prior art systems employing lamellae, it is with the construction according to the present invention generally desired to reflect as much light as possible. This means that small details and edges become of importance in the design and construction.

Therefore, in an embodiment, the two side plates further each comprise a UV light reflective element, a UV light reflective layer or a UV light reflective coating on a surface configured to face the opposite one of the two side plates in the assembled condition of the lighting system.

Thereby, absorption of UV light at the side plates is avoided and the amount of light reflected is increased considerably.

In an embodiment, the two side plates extend perpendicular to the reflector in the assembled condition of the lighting system.

Thereby the amount of light reflected is increased even further.

In an embodiment, the width of the reflector is larger than the width of the bridge component, the width of the reflector and the width of the bridge component being measured, in the assembled condition of the lighting system, in a direction between and perpendicular to mutually opposite parts of the circumferential wall of the housing.

Thereby it is ensured that the reflector is wider than and runs beneath the side plates of the bridge component and thus the reflecting elements or surfaces of the bridge component. Thereby, an infinite sharp corner or a so-called “leaky corner” is provided. If this corner was open, then light could leak into the absorbing black cavity from the bridge and would thereby no longer contribute to the output light beam. However, with the construction as described above, a defect free optical corner is provided for, which in turn increases the optical efficiency of the lighting system.

In an embodiment, the reflector is made of a material chosen from the group comprising metals, spring metals, aluminum, titanium, metallized metal sheets and metallized polymer sheets.

Such materials have been shown to have advantageous properties both in terms of reflectivity of UV light and in bendability within the elastic regime.

In an embodiment, the reflector comprises a thickness being 0.5 mm or less, 0.3 mm or less, 0.2 mm or less, or 0.1 mm or more.

Such material thicknesses are particularly advantageous when desiring to bend the material by subjecting it to a level of stress falling within the elastic regime of the material. Also, the material thickness is very important to the minimum bending radius, since the thinner the sheet material the smaller the possible minimum parabolic radius becomes.

In an embodiment, the housing comprises a parabolic surface arranged and configured to support the reflector in the assembled condition of the lighting system.

Thereby, a lighting system being particularly robust and stable, especially in terms of ensuring that the parabolic reflector is fixed in the correct shape after bending, is provided for.

In an embodiment, one or more of the holding component and the side walls of the housing is made of a UV light absorbing material.

In an embodiment, one or more of the holding component and the side walls of the housing comprises a UV light absorbing layer or coating.

These absorbing faces all have an orientation parallel to the optical axis of the lighting system. Thereby, unwanted light which may otherwise cause safety issues is absorbed by the UV absorbing surfaces.

In an embodiment, the lighting system further comprises a heat sink element on which the at least one LED light source is arranged.

The heat sink element, which preferably is a heat sink cavity or a hollow heat sink, allows convection of air to provide cooling of the optical components, and in particular the UV LED light sources and the associated electronics. Thereby, a more durable lighting system is provided.

In an embodiment, the housing comprises at least one first ventilation opening provided at a first position in the circumferential side wall and at least one second ventilation opening provided at a second position in the circumferential side wall, the second position being above the first position in a mounted condition of the lighting system, such as to enable an air flow between the at least one first ventilation opening and the at least one second ventilation opening.

Thereby an enhanced cooling of the optical components, and in particular the UV LED light sources and the associated electronics, is provided for.

In an embodiment, the lighting system further comprises a fan arranged and configured to provide an enhanced air flow between the at least one first ventilation opening and the at least one second ventilation opening.

Thereby an even further enhanced cooling of the optical components, and in particular the UV LED light sources and the associated electronics, is provided for. Furthermore, a fan is advantageous to add for instance for the visible application if a lot of light is needed.

In an embodiment, a gap is provided between the heat sink element and the side walls of the housing.

Thereby a free passage for the cooling air flowing between the at least one first ventilation opening and the at least one second ventilation opening is provided for. The gap thus in essence works as a thermal chimney. This prevents additional light leakage from the folded reflector, and further ensures multiple light reflections before it light is allowed to exit the luminaire, such that the light levels are on an acceptable low level. The system could be passively or actively cooled depending on the performance needed for the UVC LED light sources which need to be kept at relative low temperatures. The gap, or thermal chimney, ensures a proper airflow next to the heat sink. Further, the gap also provides for a space in which the fan may advantageously be arranged.

In an embodiment, the gap increases in width in a direction towards the back wall of the housing of the lighting system.

Thereby, the lighting system is made easier to mold.

In an embodiment, the width of the gap it between 6 mm and 12 mm.

Thereby sufficient room is provided around the heat sink to ensure a stable air flow between the at least one first ventilation opening and the at least one second ventilation opening. Also, an even smaller gap is possible when there are no holes on the outside. In that case the gap may be minimized just for assembly. The width of the gap may then be as small as 1 to 2 mm. Further, if 3D printing is used, it would be possible to employ straight walls. This also enables low volume high accurate optics.

In an embodiment, the lighting system further comprises at least one of a heat sink element on which the at least one LED light source is arranged, and a front wall extending opposite to and parallel with the back wall, the front wall comprising a window and a window frame, and the lighting system further comprises an optical area in which interaction between UV light and components of the lighting system occurs, and any one or more of an outer edge of the window, an outer edge of the window frame and the heat sink element is/are arranged outside of the optical area.

It is desired to minimize absorbing surfaces and prevent possible light rays being emitted in an unwanted direction. By ensuring that one or more of the outer edge of the window; and outer edge of the window frame and the heat sink element are arranged outside of the optical area it is ensured that there is no optical interaction of small edges inside the system. This helps minimizing absorbing surfaces and preventing possible light rays being emitted in an unwanted direction.

If further ensuring that all edge of the front window and the reflector connections are arranged outside the optical cavity, a low glare and/or stray light is obtained by preventing scattering on edges inside the optical beam. Thereby, UVC light scattering through the safety plane is effectively hindered when used in upper air cleaning systems.

In an embodiment, the lighting system further comprises a front wall extending opposite to and parallel with the back wall, the front wall comprising a window and a window frame, and the window being an UV transparent window, such as a window made of quartz or fused silica.

Thereby, the safety of the lighting system is increased since the possibility of touching electronic components of the lighting system is eliminated and since ingress of dust into to the cavity of the lighting system is avoided.

In an embodiment the reflector is arranged in the housing by air forming or vacuum forming.

Thereby, a simplified manufacturing process is provided for.

In an embodiment, the at least one LED light source is arranged on a substrate.

Thereby a more stable and robust construction as well as a simpler and more robust electrical supply of the at least one LED light source is provided for.

The invention further relates to a lighting system configured to disinfect air in an upper part of a space or room, such as a part of a space or room near a ceiling of the space or room, the lighting system comprising a housing comprising a back wall configured for abutment with a mounting surface and a circumferential wall extending from the back wall, at least one LED light source configured to, in operation, emitting UV light, the at least one LED light source being arranged on a substrate, a reflector configured to be arranged between the back wall and the at least one LED light source such as to reflect UV light in a direction generally away from the back wall, where the reflector is an elastically deformable reflective sheet, where the lighting system further comprises a bridge component shaped and configured to be arranged between the reflector and the at least one LED light source, where the bridge component comprises two side plates and a holding component, where the two side plates each comprise an upper edge, a lower edge and a front edge extending between the upper edge and the lower edge, the front edge being configured for abutment with the reflector in the assembled condition of the lighting system, the front edge comprising a parabolic curvature such that when the front edge and the reflector are brought into abutment during assembly of the lighting system, the reflector is provided with a parabolic curvature corresponding to that of the front edge, and where the holding component extends between and perpendicular to the side plates midways between the upper edge and the lower edge, and the holding component is configured for abutment with the reflector in the assembled condition of the lighting system.

It is noted that the invention relates to all possible combinations of features recited in the claims.

### BRIEF DESCRIPTION OF THE DRAWINGS

This and other aspects of the present invention will now be described in more detail, with reference to the appended drawings showing embodiment(s) of the invention.

FIG. 1 is a perspective view of a lighting system according to the invention.

FIG. 2 is a perspective view of the lighting system according to FIG. 1 where some parts are made transparent to show further details.

FIG. 3 is an exploded perspective view of the lighting system according to FIG. 1.

FIG. 4 is a front side vertical perspective cross-sectional view of the lighting system according to FIG. 1.

FIG. 5 is a cross-sectional side view of the lighting system according to FIG. 1.

FIG. 6 is a cross-sectional front view of the lighting system according to FIG. 1.

FIG. 7 is an enlarged perspective cross-sectional view of the detail marked VII in FIG. 6.

FIG. 8 is an enlarged cross-sectional side view of the detail marked VIII in FIG. 4.

FIG. 9 is a cross-sectional view seen in the direction IX shown in FIG. 1.

FIG. 10 shows a plot of the maximum bending stress of an aluminum sheet material as a function of the sheet thickness for a given parabolic reflector design.

FIG. 11 shows a plot of the maximum bending stress of an aluminum sheet material as a function of a design scale factor. Particularly, the fixed available sheet thickness and the given parabolic reflector from FIG. 10 is scaled towards the elastic region.

FIG. 12 shows a plot illustrating an acceptable minimum curvature of a sheet to be used as a parabolic reflector of a lighting system according to the invention.

FIG. 13 shows a plot of a simulation of the local stress inflicted to a sheet material with a thickness of 0.3 mm and illustrating curvature analyses performed to define the minimum curvature of the shape of a parabolic reflector of a lighting system according to the invention.

FIG. 14 shows a plot of a simulation the local stress inflicted to a sheet material with a thickness of 0.2 mm and used to make a parabolic reflector of a lighting system according to the invention. Particularly, FIG. 14 shows the estimated reflector deformation with a curvature plot when assembled inside the housing. This deformed shape is used in optical simulations to estimate beam artefacts.

FIG. 15 shows an intensity plot of the light output obtained using the ideal parabolic reflector without deformation.

FIG. 16 shows an intensity plot of the light output obtained using the estimated deformed shape from assembly of a reflector and taking tolerances into account.

As illustrated in the figures, the sizes of layers and regions are exaggerated for illustrative purposes and, thus, are provided to illustrate the general structures of embodiments of the present invention. Like reference numerals refer to like elements throughout.

### DETAILED DESCRIPTION

The present invention will now be described more fully hereinafter with reference to the accompanying drawings, in which currently preferred embodiments of the invention are shown. This invention may, however, be embodied in many different forms and should not be construed as limited to the embodiments set forth herein; rather, these embodiments are provided for thoroughness and completeness, and fully convey the scope of the invention to the skilled person.

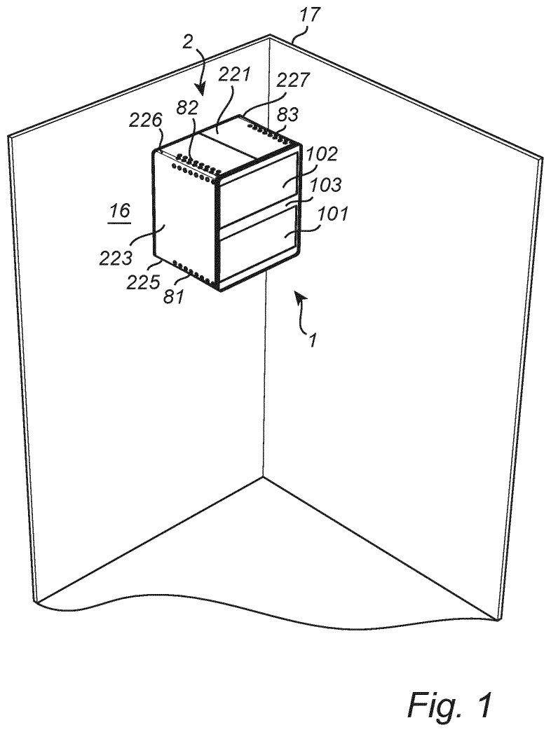

Reference is first made to FIGS. 1-3 showing a lighting system 1 according to the invention in a perspective view, in a perspective view where some parts are made transparent to show further details, and in an exploded perspective view, respectively.

Generally, and irrespectively of the embodiment, the lighting system 1 according to the invention is configured to disinfect air in an upper part of a space or room, such as a part of a space or room near a ceiling 17 of the space or room.

Generally, and irrespectively of the embodiment, the lighting system 1 comprises a housing 2 comprising a back wall 21, a LED light source 3 configured to, in operation, emit UV light, a reflector 5 configured to be arranged between the back wall 21 and the LED light source 3 such as to reflect UV light in a direction generally away from the back wall 21 and a bridge component 6 shaped and configured to be arranged between the reflector 5 and the LED light source 3.

The housing 2 comprises a back wall 21. The back wall 21 may be configured for abutment with a mounting surface. The mounting surface may for instance be a wall 16, such as a wall a space or room near a ceiling 17 of a space or a room. The housing further comprises a circumferential wall 22 extending from the back wall 21. The lighting system 1 may also be mounted via the circumferential wall 22 to a mounting surface. In the embodiment shown, the housing 2 is substantially box shaped. The circumferential wall 21 comprises an upper wall 221, a lower wall 222 extending in parallel with the upper wall 221 and two mutually parallel side walls 223 and 224 extending between the upper wall 221 and the lower wall 222. The side walls 223 and 224 may be made of a UV light absorbing material. Alternatively, the side walls 223 and 224 may comprise a UV absorbing layer or coating. The housing 2 also comprises front wall 10 acing as a light exit facet. The housing 2 may also comprise other shapes than box-shaped.

The LED light source 3 may be configured to, in operation, emit UV light comprising a component within the UVC spectrum. The LED light source 3 is arranged such that, in operation, the UV light is emitted in a direction generally towards the back wall 21 of the housing 2. The UV light may also comprise one or more of violet light of a wavelength of 420 nm or less, UVA light and UVB light. One or more such LED light sources 3 may be provided. The one or more LED light sources 3 is arranged on a substrate 4. The substrate 4 may be a printed circuit board. The LED light source 3 may alternatively be configured to, in operation, emit visible light or infrared light.

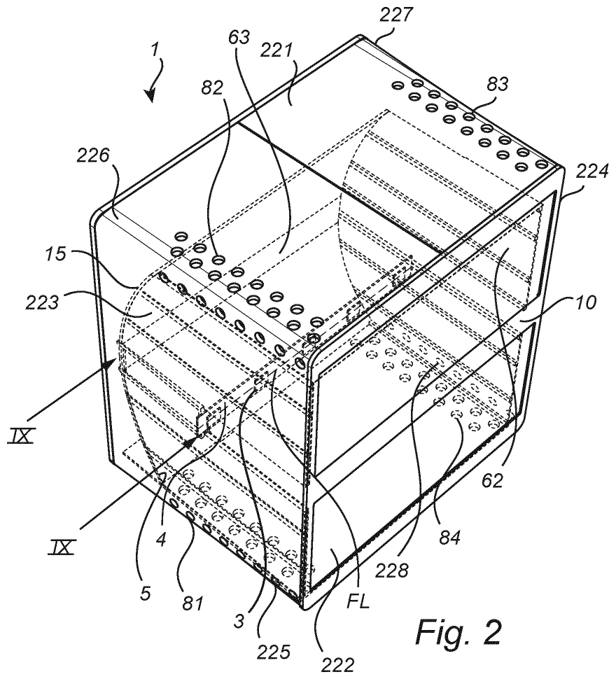

In the embodiment shown, five LED light sources 3 arranged on a line, that is a linear array of LED light sources 3, such as UV LED sources, are provided-cf. FIG. 2. A linear array of UV LEDs illuminates the parabolic reflector 5 in such a way that a strongly asymmetric beam is obtained. All radiation emitted by the LED light sources 3 is controlled by the parabolic reflector 5 and the flat reflective sides 71, 72 of the bridge component 6 described further below. The narrow beam has a FWHM in the range 0.5 degrees to 10 degrees. A preferred beam width for most applications is a FWHM in the range of 3 to 5 degrees. In a direction orthogonal to the narrow beam, the FWHM can be in the range from 90 to 150 degrees.

The reflector 5 is configured to be arranged between the back wall 21 and the at least one LED light source 3 such as to reflect UV light in a direction generally away from the back wall 21. The reflector 5 is an elastically deformable reflective sheet. The reflector 5 may be made of a material chosen from the group comprising metals, spring metals, aluminum, titanium, metallized metal sheets and metallized polymer sheets. The reflector typically comprises a thickness being 0.3 mm or less, although thicknesses of up to 0.35 or even 0.4 mm or down to 0.2 mm or even 0.1 mm are feasible.

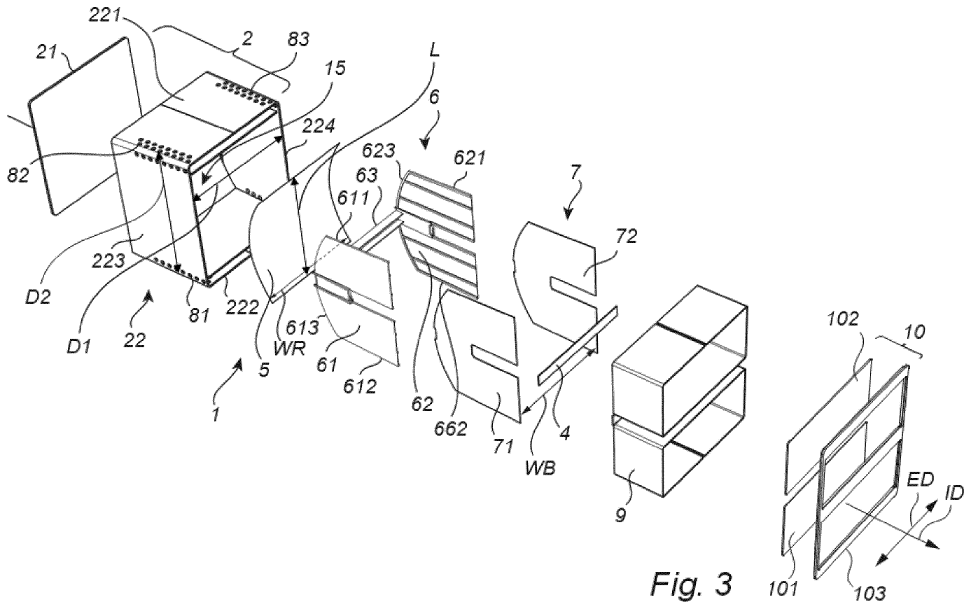

The bridge component 6 is generally shaped and configured to be arranged between the reflector 5 and the at least one LED light source 3 in such a way that during assembly of the lighting system 1 the bridge component 6 shapes the reflector 5 by bending the elastically deformable reflective sheet into a parabolic shape while subjecting the elastically deformable reflective sheet to a level of stress within the elastic regime of the elastically deformable reflective sheet.—cf. especially FIG. 2. The bridge component 6 is elongated in an elongation direction ED (cf. FIG. 3) perpendicular to a main issue direction ID (cf. FIG. 3) of the light source light. The bridge component 6 is arranged between the reflector 5 and the LED light source 3 in such a way that the reflector 5 is forced to assume a curved shape around the bridge component 6.

The way the reflector 5 is forced to assume the curved shape can, for example, be explained as follows:
The back wall 21 and the circumferential wall 22 form the housing 2 with a cavity 23 with an open side 46 opposite to the back wall 21. The bridge component 6 is to be inserted into the cavity 23. The bridge component 6 has a width WB somewhat shorter than a first distance D1 between a first pair of opposite portions of the circumferential wall 22, i.e. side walls 223, 224;
The reflector 5 initially is arranged at the open side 46 on the circumferential wall 22. The reflector 5 has a width WR just shorter than the first distance D1, and the width of the bridge component WB is in between 0.9*WR and 1*WR, here 0.95 times the width WR, and the reflector 5 has a length L which is larger than a second distance D2 between a second pair of opposite portions of the circumferential wall 22, i.e. top wall 221 and bottom wall 222 (which are oriented perpendicular to the side walls 223,224);
With the reflector 5 being initially arranged on the open side 46, the bridge portion 6 is inserted into the cavity 23 and thereby pushing the reflector 5 into the cavity 23. The reflector 5 thus is forced to assume a curved shape around the bridge component 6 by the combined working of the top wall 221 and bottom wall 222 of the circumferential wall 22, the bridge component 6 and the specified length L of the reflector 5 being larger than the second distance D2 between the top wall 221 and bottom wall 222.

The parabolic shape into which the elastically deformable reflective sheet, and thus the reflector 5, is bent follows a curve extending between the upper wall 221 and the lower wall 222 of the housing 2. The curved shape comprises a parabolic cross section in a plane perpendicular to the elongation direction ED, and a maximum level of stress imposed on the elastically deformable reflective sheet is falling within the elastic regime of the material of the elastically deformable reflective sheet. FIGS. 2 and 3 in combination show that the parabolic reflector has a focal line FL that extends in the elongation direction ED and that the light source 3 is arranged on said focal line.

In the embodiment shown, cf. especially FIG. 3, the bridge component 6 comprises two side plates 61, and 62 and a holding component 63. The side plate 61 comprises an upper edge 611, a lower edge 612 and a front edge 613 extending between the upper edge 611 and the lower edge 612. The front edge 613 is configured for abutment with the reflector 5 in the assembled condition of the lighting system 1. The front edge 613 comprises a parabolic curvature such that when the front edge 613 and the reflector 5 are brought into abutment during assembly of the lighting system 1, the reflector 5 is provided with a parabolic curvature corresponding to that of the front edge 613. Likewise, the side plate 62 comprises an upper edge 621, a lower edge 622 and a front edge 623 extending between the upper edge 621 and the lower edge 622. The front edge 623 is configured for abutment with the reflector 5 in the assembled condition of the lighting system 1. The front edge 623 comprises a parabolic curvature such that when the front edge 623 and the reflector are brought into abutment during assembly of the lighting system 1, the reflector 5 is provided with a parabolic curvature corresponding to that of the front edge 623.

The holding component 63 extends between and perpendicular to the side plates 61 and 62 midways between the upper edge 611, 621 and the lower edge 612, 622. The holding component 63 is configured for abutment with the reflector 5 in the assembled condition of the lighting system 1. The holding component 63 may be made of a UV light absorbing material. Alternatively, the holding component 63 may comprise a UV light absorbing layer or coating.

As may be seen from FIG. 3 in particular, the side plates 61 and 62 further comprise a reflective element 71 and 72, respectively. Alternatively, the side plates 61 and 62 may comprise a reflective layer or a reflective coating on a surface facing the opposite one of the side plates 62 and 61 in the mounted condition thereof. In the assembled condition of the lighting system 1, the side plates 61 and 62 extend perpendicular to the reflector 5.

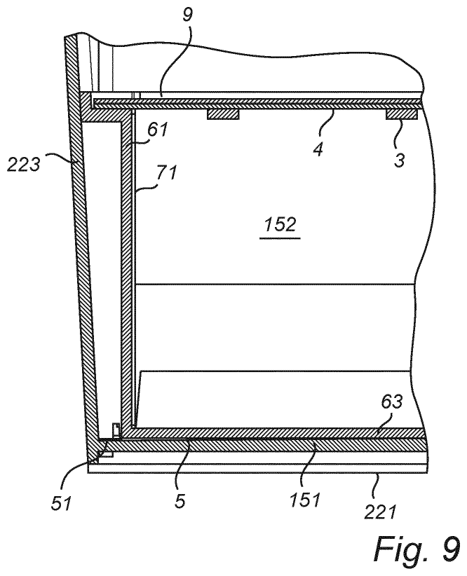

As may be seen from FIG. 3 and the enlarged view of FIG. 9 in particular, the reflector 5 comprises a width WR and the bridge component 6 comprises a width WB. In the embodiment shown, the width WR of the reflector 5 and the width WB of the bridge component 6 are measured, in the assembled condition of the lighting system 1, as the shortest distance in a direction between and perpendicular to the side walls 223 and 224 of the housing 2. In more general terms, the width WR of the reflector 5 and the width WB of the bridge component 6 may be measured, in the assembled condition of the lighting system 1, in a direction between and perpendicular to mutually opposite parts of the circumferential wall 22 of the housing 2. In any event, the width WR of the reflector 5 is chosen to be larger than the width WB of the bridge component 6. For instance, the width WR of the reflector 5 is chosen to be larger than the width WB of the bridge component 6 by an amount corresponding to at least the combined thickness of the two side plates 61 and 62 of the bridge component 6. Thereby, it is ensured that an end section 51 of the reflector 5 extends to a position closer to the side wall 223 than the reflective element 71 provided on the side plate 61. Although not shown, it is likewise ensured that an end section of the reflector 5 extends to a position closer to the opposite side wall 224 than the reflective element 72 provided on the side plate 62.

As is best seen in FIG. 3, the lighting system 1 further comprises a heat sink element 9 and a front wall 10. Both the heat sink element 9 and the front wall 10 are optional elements.

The at least one LED light source 3, and if provided the substrate 4, is arranged on the heat sink element 9 such that heat generated by the at least one LED light source 3 is conducted by the heat sink element 9 away from the bridge component 6 and the reflector 5. The heat sink 9 is made of a material efficiently conducting heat, such as a suitable metal. The holding component 63 may comprise a width being equal to or larger than the largest width of the heat sink element 9.

The front wall 10 extends opposite to and parallel with the back wall 21. The front wall 10 comprises a window 101, 102 and a window frame 103. In the embodiment shown, the window 101, 102 comprises two panels. The window 101, 102 may also comprise one panel or more than two panels. Referring also to FIG. 8, the window 101, 102 may comprise two layers, namely an outer pane 104 and an inner pane 105. The inner pane 105 is typically a quartz window. The inner pane 105 is arranged underneath the outer pane 104, that is between the outer pane 104 and the heat sink 9.

As may be seen from the enlarged view of FIG. 8, the lighting system 1 further comprises an optical area 13. The optical area 13 is the area in which interaction between UV light emitted by the LED light source 3 and components of the lighting system, such as the reflector 5 and the holding component 6, occurs. In FIG. 8, the optical area 13 is illustrated as the area below the dashed line 14. As may also be seen from FIG. 8, the outer edge 1031 of the window frame 103, the outer edge 1041 of the inner pane 104, the outer edge 1051 of the outer or quartz pane 105 and the heat sink element 9 are arranged outside of the optical area 13.

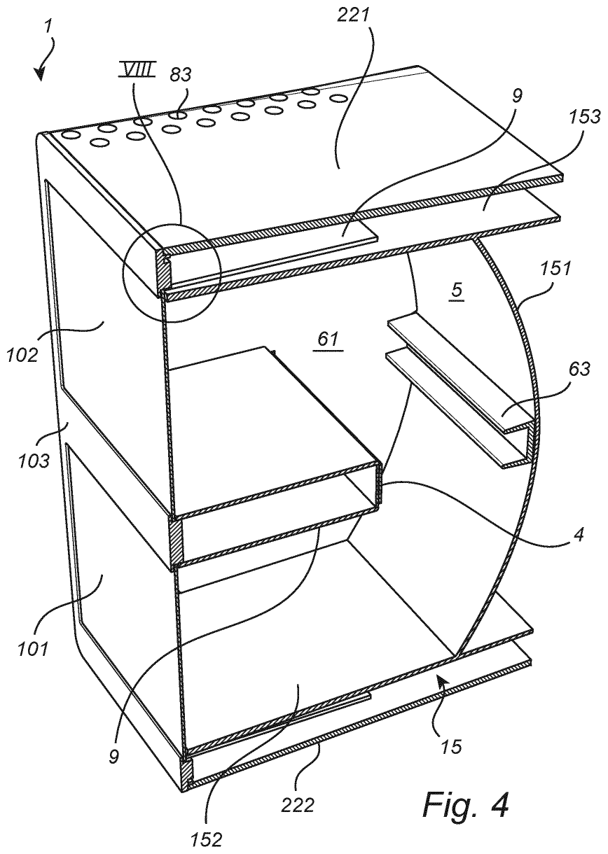

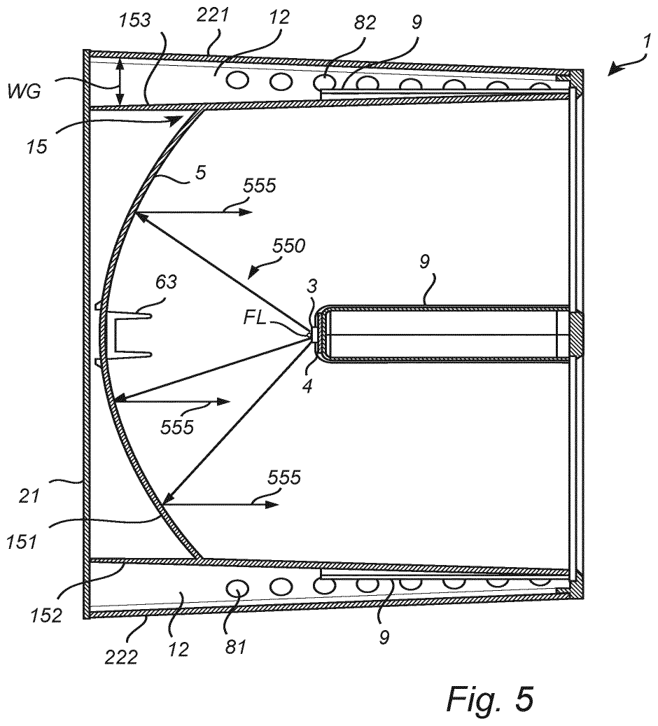

Referring now also to FIGS. 4 and 5, the housing 2 may optionally comprise a parabolic surface 151 arranged and configured to support the reflector 5 in the assembled condition of the lighting system 1. In the embodiment shown, the parabolic surface 151 is provided as a part of an insert 15. In the embodiment shown, the insert 15 further comprises mutually opposite side panels 152 and 153. The side panels 152 and 153 are configured for engagement with the heat sink 9 upon assembly of the lighting device 1. In the assembled condition of the lighting device, the side panels 152 and 153 extend adjacent to the top wall 221 and the bottom wall 222, respectively, of the housing 2. The side panels 152 and 153 may be made of a UV light absorbing material. Alternatively, the side panels 152 and 153 may comprise a UV absorbing layer or coating. It I also feasible that the side panels 152 and 153 may be omitted. As shown in FIG. 5, the light source 3 is arranged on the focal line FL of the parabolic reflector 5. During operation the light source emits a light beam 550 towards the reflector, which light beam is reflected and collimated by the reflector into a collimated beam of light rays 555 that lie mutually parallel in a direction perpendicular to the elongation direction and the main issue direction.

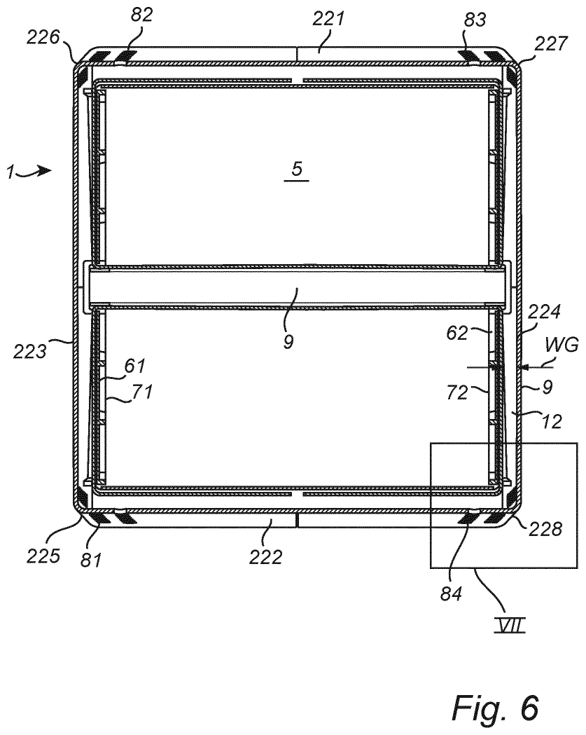

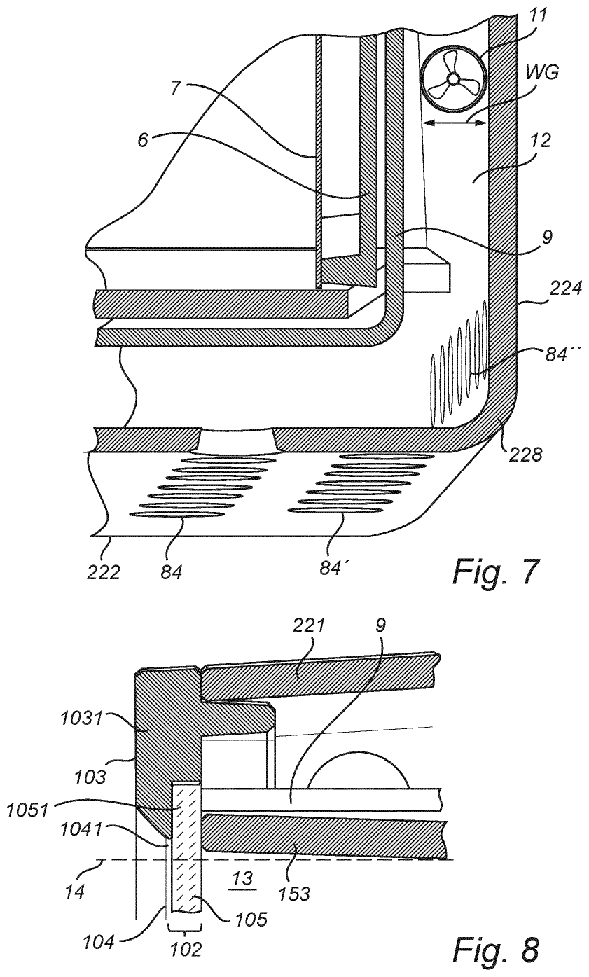

Referring now also to FIGS. 6 and 7, the housing 2 optionally comprises at least one first ventilation opening 81 provided at a first edge 225 of or at a first position in the circumferential side wall 22 and at least one second ventilation opening 82 provided at a second edge 226 or at a second position in the circumferential side wall 22. In the embodiment shown three rows of ventilation openings 81 and 82 are shown. In the embodiment shown, the second edge 226 is located above and vertically opposite to the first edge 225 in a mounted condition of the lighting system 1. In more general terms, the second position is located above the first position in a mounted condition of the lighting system 1. In any event the ventilation openings 81 and 82 are configured to enable an air flow between the first ventilation openings 81 and the second ventilation openings 82 such as to enhance the cooling of the lighting system 1.

For further enhancement of the cooling of the lighting system 1 further ventilation openings may be provided. For instance, and referring particularly to FIG. 6, in the embodiment shown there is provided at least one third ventilation opening 83 provided at a third edge 227 of or at a third position in the circumferential side wall 22 and at least one fourth ventilation opening 84 provided at a fourth edge 228 or at a second position in the circumferential side wall 22. In the embodiment shown three rows of ventilation openings 83 and 84 are shown.

Referring still to FIGS. 6 and 7, a gap 12 is provided between the heat sink element 9 and the side walls 223, 224 of the housing 2. The gap 12 acts as a passage for the air flow between the first ventilation openings 81 and the second ventilation openings 82 and/or the air flow between the third ventilation openings 83 and the fourth ventilation openings 84. The gap 12 comprises a width WG. The width WG of the gap 12 may increase in a direction towards the back wall of the housing of the lighting system-cf. for instance FIG. 5. For instance, the gap 12 may be at least 6 mm wide. For instance, the width WG of the gap 12 may increase from 6 mm to 12 mm.

Furthermore, and optionally, a fan 11 (cf. FIG. 7) may be provided. The fan 11 is configured to provide an enhanced air flow between the first ventilation openings 81 and the second ventilation openings 82 and/or an enhanced air flow between the third ventilation openings 83 and the fourth ventilation openings 84. The fan 11 may be arranged in the gap 12.

### Example 1

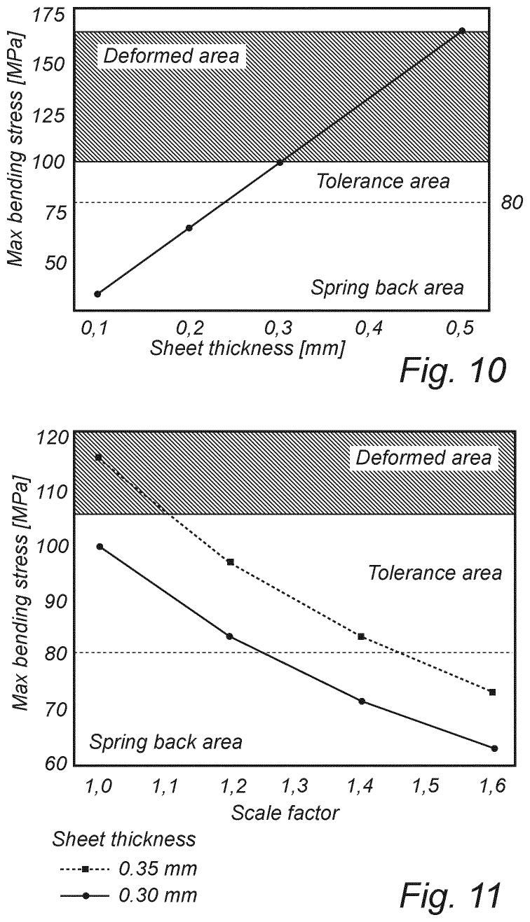

An example for further explanation of the connection between the thickness of the sheet material used for the reflector 5 and the maximum achievable bending radius will now be described with reference to FIGS. 10-13.

FIG. 10 shows a plot of the maximum bending stress of an aluminum sheet material as a function of the sheet thickness for a given parabolic design. In the present invention, we are only interested in using the elastic region. The plastic region is less accurate as bending occurs and it cannot be considered elastic.

FIG. 11 shows a plot of the maximum bending stress of an aluminum sheet material as a function of a scale factor. The scale factor is derived from the given parabolic curvature from FIG. 10 at a certain sheet thickness. The scale factor is used for optimization of the optical system towards the elastic region of the material of the reflector. This can be done by using a smaller thickness or scaling the optical system around its optical center.

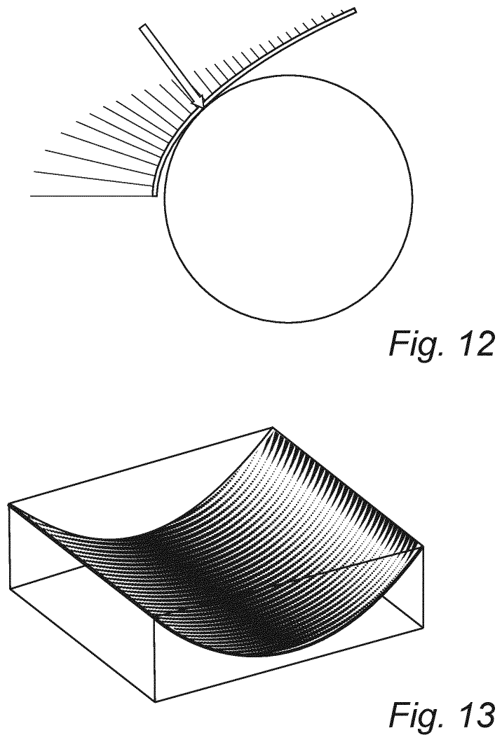

FIG. 12 shows a plot illustrating an acceptable minimum curvature of a sheet to be used as a parabolic reflector of a lighting system according to the invention. The circle in FIG. 12 shows the minimum radius possible with this specific sheet thickness and material combination. Next to the parabolic curvature this shows that the parabolic radius is still smaller then needed for this material and sheet thickness combination.

FIG. 13 shows a plot of the local stress inflicted to a sheet material with a thickness of 0.3 mm and illustrating curvature analyses performed to define the minimum curvature of the shape of a parabolic reflector of a lighting system according to the invention.

To define the minimum bending radius possible without deformation of a given sheet material used for making the reflector 5, one needs to determine the maximum bending stress or maximum strain value allowable in the sheet. Depending on the sheet thickness this results in a certain bending stress and determines if plastic deformation or elastic deformation occurs. FIG. 10 illustrates that for a constant parabolic shape and increasing sheet thickness, the maximum bending stress measured in MPa increases. FIG. 10 further shows that sheet materials of thinner thicknesses than 0.3 mm are feasible. Smaller reflectors need a thinner sheet. As small thicknesses as 0.1 mm are feasible. At such small thicknesses the sheet material has a very low bending stress but handling these sheets becomes more difficult. In other words, the lower practical limit of the material thickness is a trade-off between low bending stresses and difficult handling due to the fragility of the material increasing with smaller thicknesses.

If the sheet thickness becomes sufficiently large, in the present example over 0.3 mm, bending the sheet into the chosen parabolic shape results in the stress induced being in the plastic regime, that is inflicting plastic deformation of the sheet. The plastic regime corresponds in the graphs of FIGS. 10 and 11 to the area above the solid line.

If the sheet thickness is kept sufficiently small, in the present example below about 0.25 mm, bending the sheet into the chosen parabolic shape results in the stress induced being in the elastic regime, that is inflicting elastic deformation of the sheet. On the other hand, an ultra-thin sheet is also very difficult to handle and more difficult to polish to a high reflective quality. For handling and polishing these sheets of aluminum a good balance has been found at a sheet thickness of about 0.3 mm. The elastic regime corresponds in the graphs of FIGS. 10 and 11 to the area below the dotted line.

The tolerance zone, in the graphs of FIGS. 10 and 11 illustrated as the area between the solid line and the dotted line, illustrates the tolerance stemming from the heat treatment process of the sheet and the rolling of the sheet, that is basically the full sheet production process. Inflicting bending stresses falling within this tolerance area has been shown to be one of the main problems connected with obtaining a highly accurate UVC reflector. Therefore, bending stresses falling within this tolerance area need to be avoided.

In FIG. 11 a plot of the maximum bending stress in MPa as a function of a design scaling factor corresponding to the minimum sheet thickness normally available at suppliers is shown. In practice, a sheet thickness tolerance (curve shown with dashed line) may be included to secure a maximum use of elastic sheet area.

FIGS. 12 and 13 illustrate a minimum curvature check. FIG. 12 indicates the minimum acceptable curvature by means of the arrow inserted. FIG. 13 illustrates an exemplary simulation of a specific curvature showing the local bending stress inflicted to define the minimum curvature of the sheet. In FIG. 13, darker areas corresponds to a higher level of bending stress being inflicted.

For example, for a material with a thickness of 0.3 mm having undergone heat treatment this results in a minimum parabolic radius of 145 mm (or a diameter of 290 mm). Changing values and material properties may lead to a smaller parabolic reflector.

### Example 2

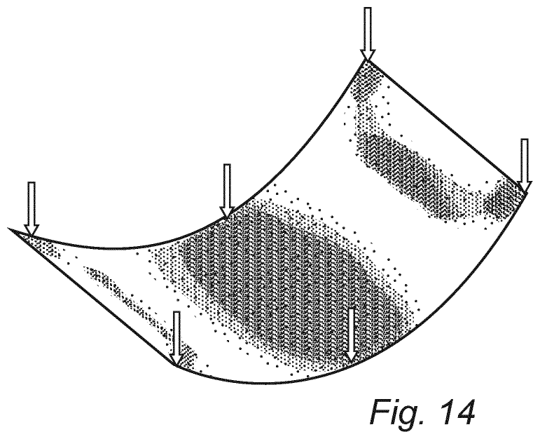

A further example will be described below with reference to FIGS. 14-16. FIG. 14 shows a curvature plot that indicates the deformed shape of the reflector in an assembled stage with bridge and housing. Variation is estimated from an assembly model to be 0.2 and will lead with a sheet of a thickness of 0.3 mm to obtain the deformation profile shown.

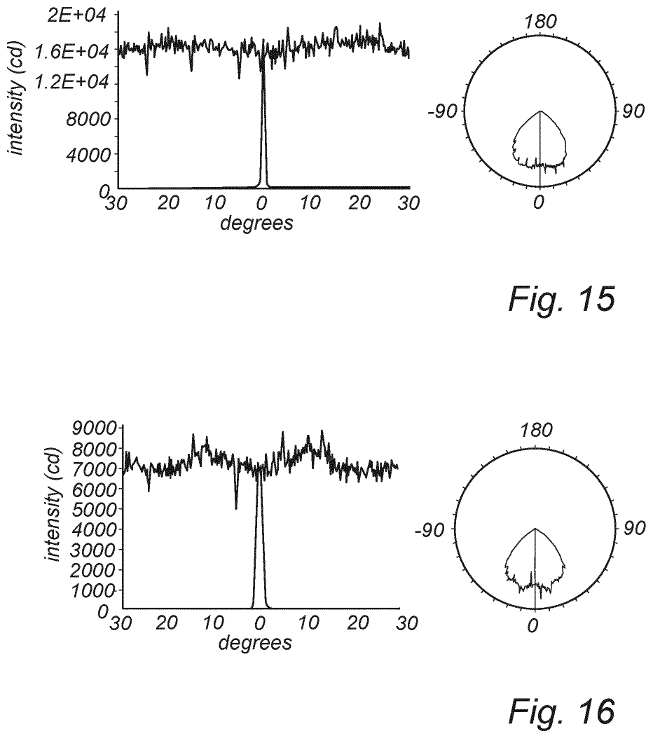

FIG. 15 shows the ideal intensity plot of the parabolic shape simulated upon in FIG. 14.

FIG. 16 shows the intensity plot of a deformed parabolic shape as simulated in FIG. 14.

As mentioned further above according to the invention only elastic deformation is used to bend the sheet material to form the reflector 5. This means that no elongation of the material is caused, and that the highest reflective performance of the material is kept or preserved. If the material used is fully elastic then there is no final deformation. This is the case for metals, which have a straight elastic region. However, due to the connection points of the bridge and the housing (see the arrows in FIG. 14) there will still be some deformation of the sheet material, albeit on a much lower level as compared to the prior art. For instance, there may be some deformation of the sheet material if the material used is not 100% elastic but rather close to 100% such as for polymer sheets. A final deformation of 0.2 mm is estimated from stress simulation as shown in FIG. 14. Due to the connection points at the bridge and end points (see the arrows in FIG. 14) a slight deformed shape in a direction other than the main bending direction occurs. The effect is that the parabolic shape is a bit too narrow. Including the deformation, however, the light beam may be widened slightly to compensate for this effect. Starting with a too narrow beam, such as between 2 and 4 degrees, makes this system ideal for optimization to get to the most ideal beam for this application. This may be done by placing the LED light sources slightly off the focal line. Overall, however, the beam still should be widened to what is ideal for tolerancing and tuning. This makes the system very robust for tolerances.

In FIG. 14, darker areas corresponds to a higher level of curvature, or in other words parabolic deviation. The ideal beam is created at the sides, and more towards the center there is some deformation of the reflector and widen the beam towards the needed beam. In other words, this means that the sides are ideal while the center is a bit deformed due to the elastic stresses. Tuning the beam could also be done by placing the LED light sources more towards the center or towards the side wall.

Adding these tolerances into optical simulation, the results shown in FIG. 15 (no tolerances included) and FIG. 16 (tolerances included) are obtained. As may be seen, the tolerances have only a limited impact on the total result. This means that the lighting system according to the invention with such an elastic sheet reflector 5 is cost competitive with the current upper air disinfection systems, and further has a smaller form factor, and is very robust towards tolerances.

The person skilled in the art realizes that the present invention by no means is limited to the preferred embodiments described above. On the contrary, many modifications and variations are possible within the scope of the appended claims. For example, a lighting system according to the invention may also be used as a visible lighting system or infrared lighting system, that is with LED light sources emitting visible or infrared light. Applications would in this case be for instance is on stage lighting or horticultural lighting if it is desired to locally illuminate plants and large areas where a ultra-narrow high efficient beam is needed. This projects a narrow low glare beam with very little stray light.

Additionally, variations to the disclosed embodiments can be understood and effected by the skilled person in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage.

1. A lighting system, the lighting system comprising:
a housing comprising a back wall and a circumferential wall extending from the back wall and forming a cavity of the housing,
at least one LED light source configured to, in operation, emit light source light,
a reflector having a reflector width in an elongation direction, the reflector being arranged between the back wall and the at least one LED light source such as to reflect the light source light as collimated light in a main issue direction generally away from the back wall, wherein
the reflector is an elastically deformable reflective sheet, and
wherein the lighting system further comprises a bridge component having a bridge width in the elongation direction perpendicular to the main issue direction,
wherein the light source, the reflector, and the bridge component are arranged in the cavity of the housing,
wherein the bridge component is arranged between the reflector and the at least one LED light source in such a way that the reflector is forced to assume a curved shape around the bridge component, said curved shape comprising a parabolic cross section in a plane perpendicular to the elongation direction, wherein a maximum level of stress imposed on the elastically deformable reflective sheet is falling within the elastic regime of the material of the elastically deformable reflective sheet.

2. The lighting system as claimed in claim 1, wherein the reflector has a focal line in the elongation direction and wherein the light source is extending along the elongation direction on the focal line of the reflector.

3. The lighting system according to claim 1, wherein the reflector is bend around the bridge component over the full bridge width of the bridge component, wherein 0.9*WR<=WB<=WR.

4. A lighting system according to claim 1, wherein the lighting system is configured to disinfect air in an upper part of a space or room, such as a part of a space or room near a ceiling of the space or room, and wherein the at least one LED light source is configured to, in operation, emit one or more of UV light source light and violet light source light.

5. A lighting system according to claim 1, wherein the circumferential wall comprises an upper wall, a lower wall extending in parallel with the upper wall and two mutually parallel side walls extending between the upper wall and the lower wall, and
wherein the curved shape into which the elastically deformable reflective sheet is bent follows a curve extending between the upper wall and the lower wall.

6. A lighting system according to claim 1, wherein the bridge component comprises two side plates and a holding component, wherein
the two side plates each comprise an upper edge, a lower edge and a front edge extending between the upper edge and the lower edge, the front edge being configured for abutment with the reflector in the assembled condition of the lighting system, the front edge comprising a parabolic curvature such that when the front edge and the reflector are brought into abutment during assembly of the lighting system, the reflector is provided with a parabolic curvature corresponding to that of the front edge, and wherein
the holding component extends between and perpendicular to the side plates midways between the upper edge and the lower edge, and the holding component is configured for abutment with the reflector in the assembled condition of the lighting system.

7. A lighting system according to claim 6, wherein the two side plates one or more of:
further each comprise a UV light reflective element, a UV light reflective layer or a UV light reflective coating on a surface configured to face the opposite one of the two side plates in the assembled condition of the lighting system, and
extend perpendicular to the reflector in the assembled condition of the lighting system.

8. A lighting system according to claim 1, wherein the width of the reflector is larger than the width of the bridge component, the width of the reflector and the width of the bridge component being measured, in the assembled condition of the lighting system, in a direction between and perpendicular to mutually opposite parts of the circumferential wall of the housing.

9. A lighting system according to claim 1, wherein the reflector is made of a material chosen from the group comprising metals, spring metals, aluminum, titanium, metallized metal sheets and metallized polymer sheets.

10. A lighting system according to claim 1, wherein the reflector comprises a thickness being 0.5 mm or less, 0.3 mm or less, 0.2 mm or less, or 0.1 mm or more.

11. A lighting system according to claim 1, wherein the housing comprises a parabolic surface arranged and configured to support the reflector in the assembled condition of the lighting system.

12. A lighting system according to claim 1, wherein one or more of the holding component and the side walls of the housing is made of a UV light absorbing material, or
wherein one or more of the holding component and the side walls of the housing comprises a UV light absorbing layer or coating.

13. A lighting system according to claim 1, wherein the housing comprises at least one first ventilation opening provided at a first position in the circumferential side wall and at least one second ventilation opening provided at a second position in the circumferential side wall, the second position being above the first position in a mounted condition of the lighting system such as to enable an air flow between the at least one first ventilation opening and the at least one second ventilation opening.

14. A lighting system according to claim 1, and further comprising a heat sink element on which the at least one LED light source is arranged.

15. A lighting system according to claim 1, and further comprising at least one of:
i) a heat sink element on which the at least one LED light source is arranged, and
ii) a front wall extending opposite to and parallel with the back wall, the front wall comprising a window and a window frame,
wherein the lighting system further comprises an optical area in which interaction between UV light and components of the lighting system occurs, and
wherein any one or more of an outer edge of the window, an outer edge of the window frame and the heat sink element are arranged outside of the optical area.
